(19) United States
(12) Patent Application Publication
(10) Pub. No.: US 2009/0036960 A1
(43) Pub. Date: Feb. 5, 2009

(54) Icey Wrap

(76) Inventor: Scott Christopher Blair, Liberty Lake, WA (US)

Correspondence Address: Scott Blair, P.O. Box 309, Liberty Lake, WA, 99019, US

(21) Appl. No.: 11/773,527

(22) Filed: Jul. 30, 2007

(51) Int. Cl. A61F 7/10

(52) U.S. Cl. 607/109

(57) ABSTRACT

ICEY WRAP can be used primarily during many outdoor activities in hot climates to help keep cool. Ice is used as the primary cooling medium. As ice melts in the internal compartment, water is absorbed by polymer crystals in the lower chamber of the wrap, is needed to continue keeping the wrap cool. Ice is more accessible, quicker to use and can be transported in any size ice chest during outside activities. Ice is also readily available at convenience stores, fast food restaurants and other establishments where outdoor activities take place. Adding ice to the ICEY WRAP is more sanitary than putting an entire traditional neck cool into an ice chest or water container that may have other items inside. Current forms of neck ties need to be periodically soaked in cold water for 10-20 minutes or placed in a refrigerator for up to 10 to 20 minutes to keep them cold. Most outdoor activities do not allow for quick access to a refrigerator and the longer delay of cooling can hinder one's activity.

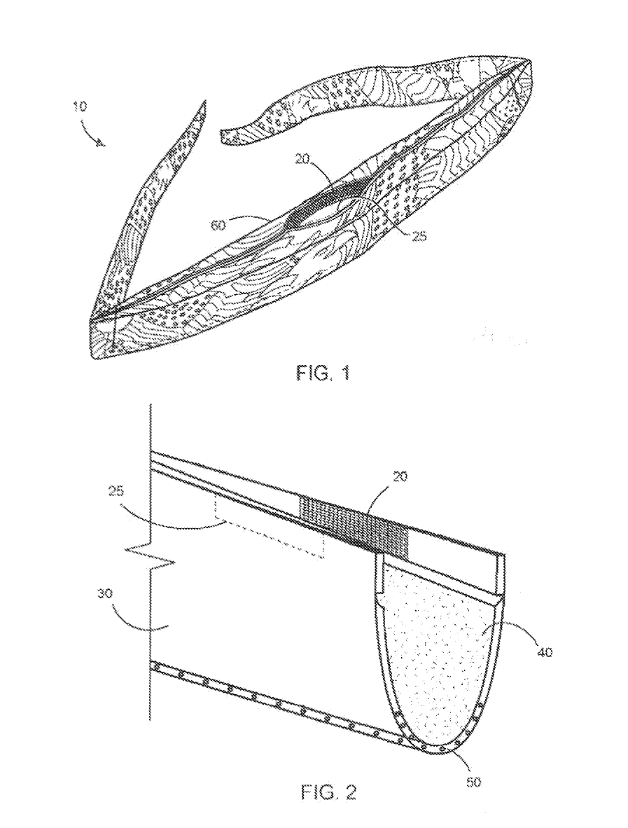

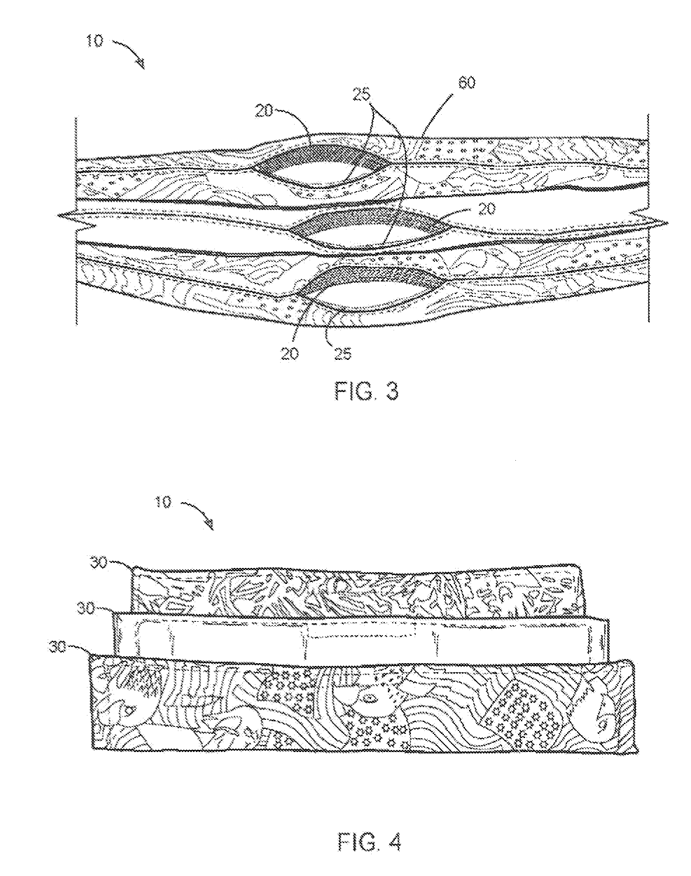

Thermal-Conducting, Absorbing, Hydrating Wearable neck wrap (titled “ICEY WRAP”) that is worn around one's neck to help keep cool. It is made of different types of fabric for design and function. An internal compartment holds ice as the cooling medium and as the ice melts, most of the water is absorbed by non-toxic polymer crystals located in the lower chamber of the neck wrap.

### DESCRIPTION

The present invention relates to a neck wrap that is worn around one's neck to help keep cool. The device is comprised of the following components:
The largest component is the fabric shell or body. It is composed of either cotton and or polyester materials (depending upon fabric patterns) and will have several different designs or appearances. The shell material holds the wrap components together with thread stitches. The two ends are about 1″ wide and taper larger to the internal compartment width of 2.5″ to 3.5″. The ends of the wrap shell are tied together around one's neck to help hold the wrap in place. There is a 4″ opening at the middle top of the wrap for inserting ice into the internal ice chamber. The opening is sealed with Velcro (hook and loop sections) or a zipper depending upon the style chosen.
The internal ice compartment is composed of terry cloth material and varies in length from 15″ to 16″ by 2.5″ to 3″ wide finished size. The terry cloth acts as a separation between the ice and the skin and also isolates the ice compartment from the lower chamber where the non-toxic water absorbing polymer crystals (Polyacrylamide, I.D. PIFL 3005 K4 Medium and Large polymer MSDS, provided by Polymers INC.) are located. These crystals slowly absorb a majority of melting ice water and retain the cool temperature at the skin contact. This cold/cool temperature at the skin can help to cool one's body through one of either conduction, convection or evaporation depending upon the surrounding environment conditions. The ice will melt normally within 10 to 15 minutes when used in hot climates for cooling thus not having an extended time of cold contact on the skin for those who may be sensitive to cold and also prevent any numbing conditions.

The ICEY WRAP internal compartment is opened at the Velcro strips (or zipper) and then filled with cubed or crushed ice. The Velcro strips are closed. The ICEY WRAP is positioned around one's neck with the Velcro access at the top. The long tapered ends are then tied comfortably around one's neck to keep the ICEY WRAP in place and in a comfortable position. A special sized buckle or hollow bead is used to help secure the tapered ends together by inserting each end of the wrap into the buckle or hold of the bead and pulling them through to a comfortable length. As the ice in the internal compartment contacts the skin areas of the neck, it provides cooling of the surfaces. When the ice melts, a little water evaporates, some water is absorbed by the terry cloth internal liner and the remainder slowly drains to the lower chamber where the polymer crystals are located. The crystals absorb most of the water and swell to a soft, moist cushion feel inside the wrap. The result between ice replenishment is cold and cool temperatures to one's skin to help you tolerate the heat from your activities. The cold and cool temperatures provided by the ICEY WRAP can also be used for aching or sore muscles and joints, injuries requiring ice and can simply be used for a neck tie or bandana if desired.

What is claimed is:

1. The Thermal-Conducting, Absorbing, Hydrating Wearable neck wrap is able to be used by individuals at variable moderate to high ambient temperatures to cool one's self through the process of evaporation and or conduction. At warm temperatures, say from 85 to 105 degrees, the individual has the choice of using tap water or refrigerated water to swell the polyacrylamide crystals in the neck wrap or one can use cubed or crushed ice to provide the cooling medium. When the temperature rises above 105 degrees to an already tested temperature of 138 degrees, the individual has the ability to use ice as the cooling medium and continually add ice as needed to maintain a workable comfort level, whereas typical neck coolers cannot add ice when needed to keep the typical neck wrap as cool. The invention, Thermal-Conducting, Absorbing, Hydrating Wearable Neck Wrap is two coolers in one.

2. The polyacrylamide crystals, located below the long terry cloth ice chamber, absorb a majority of the melting ice water and swell to a cool gel formation that forms to the wearers neck inside the base fabric. The polymer gel continues to slowly swell while absorbing the cool melting water each time ice is inserted into the ice chamber, thus the polymer gel stays cooler longer and can aid in cooling by conduction. Water that is not absorbed contacts the individual's neck and shoulder areas and aids in the evaporative cooling process. The gel does not swell up enough to prevent the individual from continually adding ice during high ambient temperatures due to natural evaporation, the absorption of melting water by the terry cloth ice chamber and the exterior base fabric.

3. Sealing of the ice chamber is accomplished by approximate ½″×4″ Velcro hook and loop strips that are stitched to the ice chamber and exterior base fabrics to avoid the potential mechanical failure of a zipper and to allow ease of sealing.
